(19) United States
(12) Patent Application Publication
(10) Pub. No.: US 2024/0293494 A1
(43) Pub. Date: Sep. 5, 2024

(54) COMPOSITION FOR IMPROVING COGNITIVE ABILITY OR MEMORY ABILITY, CONTAINING FERMENTED PERILLA FRUTESCENS EXTRACT, AND USE THEREOF

(71) Applicant: AJOU UNIVERSITY INDUSTRY-ACADEMIC COOPERATION FOUNDATION, Gyeonggi-do (KR)

(72) Inventors: Yi Sook JUNG, Gyeonggi-do (KR); Bo Kyung LEE, Gyeonggi-do (KR); Tae Ho KIM, Gyeonggi-do (KR)

(73) Assignee: AJOU UNIVERSITY INDUSTRY-ACADEMIC COOPERATION FOUNDATION, Gyeonggi-do (KR)

(21) Appl. No.: 17/922,958

(22) PCT Filed: Mar. 20, 2020
(86) PCT No.: PCT/KR2020/003846

§ 371 Date: May 4, 2023

(30) Foreign Application Priority Data
Mar. 22, 2019 (KR) 10-2019-0032982

(51) Int. Cl. A61K 36/535; A61P 25/28; C11B 9/00; D06P 1/34

(52) U.S. Cl. CPC A61K 36/535; A61P 25/28; C11B 9/0003; D06P 1/34; A61K 2236/19; D10B 2501/043; D10B 2503/02; D10B 2503/04; D10B 2503/06

(57) ABSTRACT

The present invention relates to a composition, a fiber or a fragrance composition for improving cognitive ability or memory ability, containing a fermented Perilla frutescens extract. In addition, the present invention relates to a composition for preventing, alleviating or treating cognitive ability- or memory ability-related diseases, containing a fermented Perilla frutescens extract. Furthermore, the present invention relates to a method for treating cognitive ability- or memory ability-related diseases, comprising a step of administering a fermented Perilla frutescens extract to a patient with a cognitive ability- or memory ability-related disease. Compared to an unfermented Perilla frutescens extract, a fermented Perilla frutescens extract of the present invention significantly increases cognitive ability or memory ability, thereby being effectively usable as a composition for improving cognitive ability or memory ability. Moreover, a fermented Perilla frutescens extract of the present invention can be effectively used in a composition for preventing, alleviating or treating cognitive ability- or memory ability-related diseases, or in a treatment method.

### TECHNICAL FIELD

This application claims priority to and the benefit of Korean Patent Application No. 10-2019-0032982, filed on Mar. 22, 2019, the disclosure of which is incorporated herein by reference in its entirety.

The present invention relates to a composition, a fiber and a fragrance composition for improving cognitive ability or memory ability, including a fermented extract of Perilla futescens.

In addition, the present invention relates to a composition for preventing, ameliorating or treating cognitive ability or memory ability-related disease, including a fermented extract of Perilla futescens.

In addition, the present invention relates to a method for treating cognitive ability or memory ability-related disease, including administering a fermented extract of Perilla frutescens to a patient with cognitive ability or memory ability-related disease.

### BACKGROUND ART

Modern people are exposed to more and more stimuli than in the past as the social environment changes rapidly in various ways, and rapid brain activity is required to accommodate these stimuli. Long-term learning or memory activities of adolescents, especially test takers, cause fatigue in the mind and body as well as the brain, and it may affect the healthy development of the brain. After middle age, symptoms such as memory loss or forgetfulness may appear due to a decrease in central nervous system function, and in the elderly, memory capacity decreases due to degenerative brain disease such as dementia, and social life itself becomes impossible if the degree thereof is severe. In addition, considering the impact on the family or people around, the huge social cost may be required.

Memory refers to the ability to recall past events in a conscious or unconscious state, and particularly, learning and memory are closely related and should always be considered simultaneously. Memory can be divided into unconscious nondeclarative memory, which includes skill acquisition and habits, and declarative memory, which can be consciously reproduced, such as facts and events that have occurred in the past. However, when complex behaviors such as driving or playing the piano become a habit, declarative memory can be converted into nondeclarative memory by repetition, just as it can be performed unconsciously.

Declarative memory is classified into working memory that focuses on the information given now and temporarily stores it only for a few seconds, short-term memory that lasts for hours to days, remote memory that remembers events that have passed for a long period of time and the like. Short-term memory includes the period during which memory of an ongoing event is converted into remote memory. During this period, the storage as memory is weak and it is easy to forget, but once stored as remote memory, it is not easily forgotten even if there is serious damage to the brain.

Memory can consist of three stages, and the first stage is an input step of memorizing new information or knowledge and inputting it to the brain, the second stage is a storage step of storing the information or knowledge input to the brain, and the third stage is a recall step of rethinking the stored information or knowledge. If an abnormality occurs in any one of the above three stages, phenomena in which information or knowledge cannot be accurately memorized, that is, memory deterioration or decline may occur.

The deterioration or decline in memory is caused by the necrosis of brain cells, which occurs due to various causes such as a decrease in the amount of acetylcholine due to the action of acetylcholinesterase in neurons of the brain tissue, neurotoxicity induced by β-amyloid (Aβ) and the like.

Accordingly, both surgical and pharmacological methods are currently used for the deterioration or decline in memory capacity, but since the surgical method is not easy, the pharmacological method is recommended. As the pharmacological method, inhibitors of the activity of acetylcholinesterase (tacrine, aricept, donepezil, rivastigrin, galantamine, etc.), which is an enzyme that degrades acetylcholine, have been commercialized, but rather than treating the deterioration or decline of memory ability, they only show temporary improvement in symptoms by regulating neurotransmitters. Furthermore, side effects such as hepatotoxicity, gastrointestinal disorders, cardiac bradycardia, weight loss and insomnia, and toxicity or dependence caused by long-term use have been pointed out as problems, and new therapeutics to compensate for these problems are being developed and required.

Meanwhile, Perilla frutescens is an annual plant belonging to the Lamiaceae family and is used to treat various diseases such as cough, phlegm, sore throat, indigestion, swelling, paralysis symptom, diabetes, low back pain and the like, and it is known to have antibacterial or anticancer effects.

Japanese Registered Patent No. 2004-219382 relates to a composition having a sleep promoting action and a beverage containing the same, and it discloses that the composition includes a leaf extract of Perilla frutescens, and when ingested, it has an effect of inducing sleep promoting such as prolonging sleep time. However, there has been no research or report on the effect of a Perilla frutescens extract fermented with microorganisms or a composition thereof on improving the cognitive ability or memory ability.

### DISCLOSURE

### Technical Problem

Accordingly, as a result of diligent efforts to provide effective natural substances without side effects while improving cognitive ability or memory ability, the inventors of the present invention completed the present invention by confirming that since a fermented extract of Perilla frutescens has an excellent effect on improving cognitive ability or memory ability, it can replace previously used chemical drugs for reduction in cognitive ability, memory ability or related diseases.

Accordingly, an object of the present invention is to provide a composition, a fiber or a fragrance composition for improving cognitive ability or memory ability, including a fermented extract of Perilla frutescens.

Another object of the present invention is to provide a composition for preventing, ameliorating or treating cognitive ability or memory ability-related disease, including a fermented extract of Perilla frutescens.

Still another object of the present invention is to provide a method for treating cognitive ability or memory ability-related disease, including administering a fermented extract of Perilla frutescens to a patient with cognitive ability or memory ability-related disease.

### Technical Solution

In order to achieve the above objects, the present invention provides a composition for improving cognitive ability or memory ability, including a fermented extract of Perilla frutescens.

According to a preferred exemplary embodiment of the present invention, the fermented extract may be extracted from any one or more parts selected from the group consisting of leaves, stems, flowers, fruits and whole plants of Perilla frutescens.

According to a preferred exemplary embodiment of the present invention, the fermented extract may be obtained by extracting with water, an organic solvent or a mixture thereof as a solvent and then fermenting with a fermentation strain.

According to a preferred exemplary embodiment of the present invention, the fermentation strain may be composed of bacillus or lactobacillus.

According to a preferred exemplary embodiment of the present invention, the bacillus may be any one or more Bacillus genus bacteria selected from the group consisting of Bacillus subtilis, Bacillus thuringiensis, Bacillus licheniformis, Bacillus amyloliquefaciens, Bacillus stearothermophilus, Bacillus coagulans, Bacillus longum, Bacillus pumilus, Bacillus brevis, Bacillus circulans and Bacillus polymyxa; and wherein the lactobacillus may be any one or more Lactobacillus genus bacteria selected from the group consisting of Lactobacillus acidophilus, Lactobacillus casei, Lactobacillus gasseri, Lactobacillus bulgaricus, Lactobacillus helveticus, Lactobacillus fermentum, Lactobacillus paracasei, Lactobacillus plantarum, Lactobacillus reuteri, Lactobacillus rhamnosus and Lactobacillus salivarius.

According to a preferred exemplary embodiment of the present invention, the fermentation may be performed at 5° C. to 80° C. for 30 minutes to 10 days.

According to a preferred exemplary embodiment of the present invention, the composition may be any one or more compositions selected from the group consisting of a food composition, a cosmetic composition, a dye composition, a pharmaceutical composition and a quasi-drug composition.

In addition, the present invention provides a fiber for improving cognitive ability or memory ability, including a fermented extract of Perilla frutescens.

According to a preferred exemplary embodiment of the present invention, the fiber may be dyed with a fermented extract of Perilla frutescens.

According to a preferred exemplary embodiment of the present invention, the fiber may be included in any one or more products selected from the group consisting of bedding, clothing, curtains, carpets, indoor shoes, towels, wallpaper, indoor cover products made of textile materials, dolls and eye masks.

According to a preferred exemplary embodiment of the present invention, the fragrance composition may be included in any one or more products selected from the group consisting of perfumes, scented candles, oils, aromatics, detergents and external skin preparations.

In addition, the present invention may provide a pharmaceutical composition for preventing or treating cognitive ability or memory ability-related disease, including a fermented extract of Perilla frutescens.

In addition, the present invention may provide a health functional food composition for preventing or ameliorating cognitive ability or memory ability-related disease, including a fermented extract of Perilla frutescens.

According to a preferred exemplary embodiment of the present invention, the cognitive ability or memory ability-related disease may be degenerative brain disease or dementia.

In addition, the present invention may provide a method for treating cognitive ability or memory ability-related disease, including administering a fermented extract of Perilla frutescent to a patient with cognitive ability or memory ability-related disease.

As described above, drugs used for the deterioration or decline in cognitive ability or memory ability in the related art have limitations in that side effects such as human internal organ disorders, toxicity or dependence occur. In order to replace the above drugs, the development of a composition capable of improving cognitive ability or memory ability by using plant-derived natural products is required.

A fermented extract of Perilla frutescens according to the present invention is fermented by fermenting the extract derived from Perilla frutescens using various microorganisms such as bacillus and the like, and since it has an effect of significantly improving cognitive ability or memory ability, it may be used as a composition for improving cognitive ability or memory ability and furthermore as a composition or therapeutic method for preventing, ameliorating or treating cognitive ability or memory ability-related disease.

As used herein, the term “fermented extract of Perilla frutescens” means that a Perilla frutescens extract is first prepared and then the extract is fermented by using a fermentation strain.

As used herein, the term “cognitive ability (cognition)” is an ability to discern and recognize objects, and it is a concept including spatial cognitive ability, thinking ability, comprehension, computation ability, learning ability, judgment ability, concentration or the like. Cognitive dysfunction may occur when cognitive ability is deteriorated or decreased, and this includes mental and behavioral disorders due to brain disease, brain injury, addiction and the like, and memory decline and memory impairment due to endocrine abnormalities, metabolic and nutritional abnormalities, drugs and the like.

As used herein, the term “memory ability (memory)” is an ability to keep previous impressions or experiences in consciousness, and it is closely related to cognitive ability. In addition, when memory ability is deteriorated or decreased, memory ability disorders such as forgetfulness may occur.

As used herein, the term “cognitive ability or memory ability-related disease” is a concept including all diseases in which cognitive ability or memory ability may be deteriorated or decreased as a symptom of the corresponding disease.

As used herein, the term “improvement” refers to any effect of qualitatively or quantitatively improving cognitive ability or memory ability, or qualitatively or quantitatively alleviating diseases related to cognitive ability or memory ability.

After a fermented extract of Perilla frutescens, which was prepared by fermenting a Perilla frutescens extract with Bacillus subtilus or Lactobacillus rhamnosus (Example 1), was administered to mice whose cognitive ability or memory had been reduced by restraint or the administration of scopolamine, the Y-maze test was performed (Example 2 and Example 3), and as a result, the spontaneous alteration of the mice was significantly increased. This means that the fermented extract of Perilla frutescens according to the present invention has an effect of improving cognitive ability or memory ability.

The scopolamine is also called hyoscine, and it is used to treat motion sickness, postoperative nausea or vomiting. It may be used as an antidepressant by blocking some of the effects of acetylcholine in the nervous system, and it may be used to study the relationship between acetylcholine and cognition or memory.

Mice whose cognitive ability or memory ability is deteriorated due to the restraint or administration of scopolamine may be considered as mice in which a decline in cognitive ability or memory ability has occurred, and furthermore, in which cognitive ability or memory ability-related disease has occurred.

The Y-maze test is a test for confirming the recovery of cognitive ability or memory ability, and it may evaluate sequential behavioral ability, spatial memory ability or spatial cognitive ability.

Accordingly, the present invention may provide a composition, a fiber or a fragrance composition for improving cognitive ability or memory ability, including a fermented extract of Perilla frutescens.

Perilla frutescens is also called perilla and is known to have antibacterial or anticancer effects. It is also used in rice dishes such as California rolls or sushi, and is also used as a dye to color food or dye fabrics.

In order to prepare a fermented extract of Perilla frutescens, first, a Perilla frutescens extract is prepared. It may be extracted from the above-ground parts or whole plants of the leaves, stems, flowers or fruits of Perilla frutescens, but is preferably extracted from the above-ground parts of the leaves, stems, flowers or fruits of Perilla frutescens. The extraction solvent may be water, an organic solvent or a mixture thereof, but preferably, water or a lower alcohol of C₁ to C₄ may be used, and more preferably, water, methanol, ethanol or propanol may be used, and most preferably, water is used as a solvent (Example 1).

For the fermentation bacterial strain that can be used to ferment the Perilla frutescens extract, bacillus or lactobacillus may be used, and it is preferable that the bacillus belongs to the genus Bacillus and the lactobacillus belongs to the genus Lactobacillus for increasing the effect of improving cognitive ability or memory ability.

The bacillus may be any one or more Bacillus genus bacteria selected from the group consisting of Bacillus subtilis, Bacillus thuringiensis, Bacillus licheniformis, Bacillus amyloliquefaciens, Bacillus stearothermophilus, Bacillus coagulans, Bacillus longum, Bacillus pumilus, Bacillus brevis, Bacillus circulans and Bacillus polymyxa.

In addition, the lactobacillus may be any one or more Lactobacillus genus bacteria selected from the group consisting of Lactobacillus acidophilus, Lactobacillus casei, Lactobacillus gasseri, Lactobacillus bulgaricus, Lactobacillus helveticus, Lactobacillus fermentum, Lactobacillus paracasei, Lactobacillus plantarum, Lactobacillus reuteri, Lactobacillus rhamnosus and Lactobacillus salivarius.

Fermentation conditions are preferably carried out for 30 minutes to 10 days at 5° C. to 80° C., more preferably carried out for 3 to 8 days at 20° C. to 40° C., and most preferably carried out for 4 to 6 days at 22° C. to 30° C. (Example 1).

The administration route of the fermented extract of Perilla frutescens may be oral or parenteral, and in the case of parenteral administration, it may be administered by a route such as skin external, injection, transdermal or nasal, but is not limited thereto. The dosage may be administered at 0.001 mg to 10,000 mg per 1 kg, and preferably, 0.01 mg to 1,000 mg per 1 kg, but is not limited thereto.

The composition of the present invention is preferably any one or more compositions selected from the group consisting of a food composition, a cosmetic composition, a dye composition, a pharmaceutical composition and a quasi-drug composition, but is not limited thereto.

When formulated as a cosmetic composition, the content of the fermented extract of Perilla frutescens is 0.0001 to 10 wt. %, and preferably, 0.01 to 5.0 wt. %, based on the total weight of the cosmetic composition. The content of the fermented extract of Perilla frutescens is preferably more than the above minimum value so as to achieve a minimum cognitive ability or memory improvement effect, and in consideration of the applicability to various formulations and lowering the feeling of use due to excessive addition, the content of the fermented extract of Perilla frutescens is preferably below the maximum value. In this case, the content of the fermented extract of Perilla frutescens is preferably properly adjusted within the above range according to the content of ingredients contained in the formulation or cosmetic composition.

The ingredients included in the cosmetic composition of the present invention include ingredients typically used for the cosmetic composition in addition to the fermented extract of Perilla frutescens as an active ingredient, and includes, for example, a typical adjuvant such as an antioxidant, a stabilizer, a solubilizer, a vitamin, a pigment, and a flavoring agent, and a carrier.

The cosmetic composition of the present invention may be prepared into any typical formulation prepared in the art, and may be formulated into a cosmetic such as, for example, softening lotion, astringent lotion, nourishing lotion, nourishing cream, massage cream, essence, eye cream, eye essence, cleansing cream, cleansing foam, cleansing water, packs, gel, powder, body lotion, body cream, body oil and body essence.

When the formulation of the present invention is a paste, a cream or a gel, an animal oil, a vegetable oil, a wax, paraffin, starch, tragacanth, a cellulose derivative, a polyethylene glycol, silicone, bentonite, silica, talc, zinc oxide or the like may be used as a carrier ingredient.

When the formulation of the present invention is a powder or a spray, lactose, talc, silica, aluminum hydroxide, calcium silicate or a poly amide powder may be used as the carrier ingredient, and particularly, when the formulation of the present invention is a spray, the formulation may additionally include a propellant such as chlorofluorohydrocarbon, propane/butane or dimethyl ether.

When the formulation of the present invention is a solution or an emulsion, a solvent, a solubilizer or an emulsifier is used as the carrier ingredient, and examples thereof include water, ethanol, isopropanol, ethyl carbonate, ethyl acetate, benzyl alcohol, benzyl benzoate, propylene glycol, 1,3-butylglycol oil, glycerol aliphatic esters, polyethylene glycol or fatty acid esters of sorbitan.

When the formulation of the present invention is a suspension, a liquid diluent such as water, ethanol or propylene glycol, a suspension such as ethoxylated isostearyl alcohol, polyoxyethylene sorbitol ester and polyoxyethylene sorbitan ester, microcrystalline cellulose, aluminum metahydroxide, bentonite, agar, tragacanth or the like may be used as the carrier ingredient.

When the formulation of the present invention is a surfactant-containing cleanser, an aliphatic alcohol sulfate, an aliphatic alcohol ether sulfate, sulphosuccinic acid monoester, isethionate, an imidazolinium derivative, methyltaurate, a sarcosinate, fatty acid amide ether sulfate, alkylamido betaine, an aliphatic alcohol, fatty acid glyceride, fatty acid diethanolamide, a vegetable oil, a lanolin derivative, an ethoxylated glycerol fatty acid ester or the like may be used as the carrier ingredient.

The fiber of the present invention meals all fibers that are produced by using the fermented extract of Perilla frutescens as a raw material, are dyed by using as a dye or are attached to the surface of a fiber by spraying and the like, thereby exhibiting the effect of improving cognitive ability or memory ability, and it is preferably a fiber dyed by using the fermented extract of Perilla frutescens as a dye.

The fiber may be used in all products that can affect the living environment, and it is preferably included in any one or more products selected from the group consisting of bedding, clothing, curtains, carpets, indoor shoes, towels, wallpaper, indoor cover products made of textile materials, dolls and eye masks, but the present invention is not limited thereto.

The bedding refers to bedlinen and a sleeping pad (blanket, mattress), a pillow, a cushion or the like required for sleeping, and the clothing refers to pajamas, underwear or socks that can be worn when sleeping, and the indoor cover products made of textile materials mean covers made of textile materials used to cover and store furniture such as chairs or desks, decorations, utensils and the like for reasons such as contamination or other reasons, but the present invention is not limited thereto.

The fragrance of the present invention refers to all materials used to produce a scent, such as an aroma.

The fragrance composition may be included without limitation as long as it is a product used for scenting while exhibiting a cognitive ability or memory improving effect due to the fermented extract of Perilla frutescens, and preferably, it may be included in any one or more products selected from perfumes, scented herbs, oils, fragrances, detergents and skin external preparations.

The skin external preparations include all products necessary for bathing, such as soap, face wash or a bathing agent, but the present invention is not limited thereto.

The amount of the fermented extract of Perilla frutescens included in the products may be appropriately adjusted and included in order to achieve the desired effects according to conventional techniques in the art.

In addition, the present invention may provide a pharmaceutical composition for preventing or treating cognitive ability or memory ability-related disease, including a fermented extract of Perilla frutescens.

Since the fermented extract of Perilla frutescens is the same as that used in the composition for improving cognitive ability or memory ability, the description thereof is replaced with the above-described description.

The cognitive ability or memory ability-related disease is a concept including all diseases in which cognitive ability or memory ability may be deteriorated or declined as a symptom of the corresponding disease, and may preferably be degenerative brain disease or dementia.

The degenerative brain disease refers to a disease occurring in the brain among degenerative diseases that occur with aging. It is a concept including all disease that can manifest the deterioration or decline of cognitive ability or memory ability as symptoms such as cerebral infarction, stroke, Alzheimer's disease, frontotemporal lobar degeneration (FTLD), Pick's disease, corticobasal degeneration (CBD), progressive supranuclear palsy (PSP), Parkinson's disease or Huntington's disease.

The dementia may be a concept including all types of dementia that occur by various causes such as degenerative brain disease, cerebrovascular dementia, AIDS-induced dementia, neuroinflammatory dementia, dementia with Lewy Bodies (DLB), multi-infarct dementia (MID), spinal cord injury or head injury.

The pharmaceutical compositions of the present invention may be in various oral or parenteral preparations. When the composition is formulated, the composition may be prepared by using one or more buffers (e.g., saline or PBS), antidiabetic agents, bacteriostats, chelating agents (e.g., EDTA or glutathione), fillers, extenders, binders, adjuvants (e.g., aluminum hydroxide), suspensions, thickeners, wetting agents, disintegrants or surfactants, diluents or excipients.

Examples of a solid preparation for oral administration include a tablet, a pill, a powder, a granule, a capsule and the like, and the solid preparation is prepared by mixing one or more compounds with at least one or more excipients, for example, starch (including corn starch, wheat starch, rice starch, potato starch, etc.), calcium carbonate, sucrose, lactose, dextrose, sorbitol, mannitol, xylitol, erythritol maltitol, cellulose, methyl cellulose, sodium carboxymethylcellulose and hydroxypropyl methylcellulose, gelatin or the like. For example, a tablet or a sugar tablet may be obtained by blending an active ingredient with a solid excipient, pulverizing the resulting blend, adding a suitable auxiliary agent thereto and then processing the same into a granular mixture. Further, in addition to a simple excipient, lubricants such as magnesium stearate and talc are also used.

A liquid preparation for oral administration corresponds to a suspension, a liquid for internal use, an emulsion, a syrup and the like, and the liquid preparation may include, in addition to water and liquid paraffin which are simple commonly used diluents, various excipients, for example, a wetting agent, a sweetener, a flavoring agent, a preservative or the like. In addition, depending on some cases, cross-linked polyvinyl pyrrolidone, agar, alginic acid, sodium alginate or the like may be added as a disintegrant, and an anti-coagulant, a lubricant, a wetting agent, a flavoring agent, an emulsifier, an antiseptic and the like may be additionally added.

Preparations for parenteral administration include an aqueous sterile solution, a non-aqueous solvent, a suspension solvent, an emulsion, a freeze-dried preparation, a suppository or the like. As the non-aqueous solvent and the suspension solvent, it is possible to use propylene glycol, polyethylene glycol, a vegetable oil such as olive oil, an injectable ester such as ethyl oleate and the like. As a base of the suppository, it is possible to use Witepsol, Macrogol, Tween 61, cacao butter, laurin fat, glycerol, gelatin and the like.

The pharmaceutical composition of the present invention may be administered orally or parenterally, and when administered parenterally, it may be formulated in the form of a preparation for external application to the skin; an injection administered intraperitoneally, intrarectally, intravenously, intramuscularly, subcutaneously, or intracerebroventricularly, or via cervical intrathecal injection; a transdermal administration agent; or a nasal inhaler according to a method known in the art.

The injection must be sterilized and protected from contamination by microorganisms such as bacteria and fungi. Examples of a suitable carrier for injection may be, but are not limited to, a solvent or a dispersion medium including water, ethanol, polyols (e.g., glycerol, propylene glycol, liquid polyethylene glycol, etc.), mixtures thereof and/or vegetable oils. More preferably, as a suitable carrier, it is possible to use an isotonic solution such as Hank's solution, Ringer's solution, triethanolamine-containing phosphate buffered saline (PBS) or sterile water for injection, 10% ethanol, 40% propylene glycol, and 5% dextrose and the like. In order to protect the injection from microbial contamination, various antimicrobial agents and antifungal agents such as paraben, chlorobutanol, phenol, sorbic acid and thimerosal may be additionally included. Furthermore, in most cases, the injection may additionally include an isotonic agent such as sugar or sodium chloride.

Examples of the transdermal administration agent include a form such as an ointment, a cream, a lotion, a gel, a solution for external use, a paste, a liniment and an aerosol. The transdermal administration as described above means that the effective amount of an active ingredient contained in a pharmaceutical composition is delivered into the skin via local administration thereof to the skin.

In the case of a preparation for inhalation, the extract used according to the present invention may be conveniently delivered in the form of an aerosol spray from a pressurized pack or a nebulizer using a suitable propellant, for example, dichlorofluoromethane, trichlorofluoromethane, dichlorotetrafluoroethane, carbon dioxide or other suitable gases. In the case of the pressurized aerosol, a dosage unit may be determined by providing a valve for transferring a metered amount. For example, a gelatin capsule and a cartridge for use in an inhaler or insufflator may be formulated so as to contain a powder mixture of a compound and a suitable powder base such as lactose or starch. Formulations for parenteral administration may be any formulation generally known in pharmaceutical chemistry.

The pharmaceutical composition of the present invention is administered in a pharmaceutically effective amount, and the pharmaceutically effective amount refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, factors including simultaneously used drugs and other factors well known in the medical field. The composition of the present invention may be administered as an individual therapeutic agent or in combination with other therapeutic agents, may be administered sequentially or simultaneously with therapeutic agents in the related art, and may be administered in a single dose or multiple doses. That is, the total effective amount of the composition of the present invention may be administered to a patient in a single dose or may be administered by a fractionated treatment protocol, in which multiple doses are administered over a long period of time. It is important to administer the composition in a minimum amount that can obtain the maximum effect without any side effects, in consideration of all the aforementioned factors, and this amount may be easily determined by a person skilled in the art.

A dosage of the pharmaceutical composition of the present invention varies according to the body weight, age, gender and health status of a patient, diet, administration time, administration method, excretion rate and the severity of a disease. A daily dosage thereof may be administered parenterally in an amount of preferably 0.01 to 200 mg, and more preferably, 0.1 mg to 120 mg per 1 kg of body weight a day based on a fermented extract of Perilla frutescens, and a daily dosage thereof may be administered orally in a single dose or multiple doses in an amount of preferably 0.01 to 200 mg, and more preferably 0.01 to 20 mg per 1 kg of body weight a day based on the fermented extract of Perilla frutescens according to the present invention. However, since the effective amount may be increased or decreased depending on the administration route, the severity of obesity, gender, body weight, age and the like, the dosage is not intended to limit the scope of the present invention in any way.

The composition of the present invention may be used either alone or in combination with surgery, radiation therapy, hormone therapy, chemotherapy and methods using a biological response modifier.

The pharmaceutical composition of the present invention may also be provided in the form of an external preparation including the fermented extract of Perilla frutescens as an active ingredient. When the pharmaceutical composition for cognitive ability or memory ability-related disease according to the present invention is used as a preparation for external application to the skin, the pharmaceutical composition may additionally contain auxiliary agents typically used in the dermatology field, such as any other ingredients typically used in the preparation for external application to the skin, such as a fatty material, an organic solvent, a solubilizing agent, a thickener and a gelling agent, a softener, an antidiabetic agent, a suspending agent, a stabilizer, a foaming agent, a fragrance, a surfactant, water, an ionic emulsifier, a non-ionic emulsifier, a filler, a metal ion blocking agent, a chelating agent, a preservative, a vitamin, a blocking agent, a wetting agent, an essential oil, a dye, a pigment, a hydrophilic active agent, a lipophilic active agent or a lipid vesicle. In addition, the ingredients may be introduced in an amount generally used in the dermatology field

When the pharmaceutical composition for cognitive ability or memory ability-related disease according to the present invention is provided as an external skin preparation, it may be formulated as an ointment, a patch, a gel, a cream or a spray, but the present invention is not limited thereto.

In addition, the present invention may provide a health functional food composition for preventing or ameliorating cognitive ability or memory ability-related disease, including a fermented extract of Perilla frutescens.

Since the fermented extract of Perilla frutescens is the same as that used in the composition for improving cognitive ability or memory ability, the description thereof is replaced with the above-described description.

Since the cognitive ability or memory ability-related disease is the same as that targeted by the pharmaceutical composition, the description thereof is replaced with the above-described description.

The type of health functional food is not particularly limited. Examples thereof include drinks, meats, sausages, bread, biscuits, rice cake, chocolate, candies, snacks, confectioneries, pizza, instant noodles, other noodles, gum, dairy products including ice cream, various soups, drinking water, alcoholic beverages and vitamin complexes, milk products and dairy products and the like, and include all health functional foods in a typical sense.

The fermented extract of Perilla frutescens according to the present invention may be added as is to food or may be used together with other foods or food ingredients and may be appropriately used by a typical method. The mixing amount of the active ingredient may be suitably determined depending on its purpose of use (for prevention or amelioration). In general, the amount of the compound in the health food may be 0.1 to 90 parts by weight of the total food weight. However, in the case of long-term intake for the purpose of health and hygiene or for the purpose of controlling health, the amount may be equal to or less than the above range, and the effective ingredient may be used in an amount equal to or more than the above range due to no problem in terms of safety.

Other ingredients are not particularly limited, except that the health functional beverage composition of the present invention contains the compound of the present invention as an essential ingredient at an indicated ratio, and the health functional beverage composition of the present invention may contain various flavoring agents like a typical beverage, natural carbohydrates and the like as an additional ingredient. Examples of the above-described natural carbohydrates include typical sugars such as monosaccharides, for example, glucose, fructose and the like; disaccharides, for example, maltose, sucrose and the like; and polysaccharides, for example, dextrin, cyclodextrin and the like, and sugar alcohols such as xylitol, sorbitol, and erythritol. As the flavoring agent except for those described above, a natural flavoring agent (thaumatin, a stevia extract (e.g., rebaudioside A, glycyrrhizin, etc.), and a synthetic flavoring agent (saccharin, aspartame, etc.) may be advantageously used. The proportion of the natural carbohydrate is generally about 1 to 20 g, and preferably about 5 to 12 g per 100 g of the composition of the present invention.

In addition to the above, The fermented extract of Perilla frutescens according to the present invention may contain various nutrients, vitamins, minerals (electrolytes), flavoring agents such as synthetic flavoring agents and natural flavoring agents, colorants and fillers (cheese, chocolate, etc.), pectic acid and salts thereof, alginic acid and salts thereof, organic acids, protective colloid thickeners, pH adjusting agents, stabilizers, antiseptics, glycerin, alcohols and carbonating agents used in a carbonated beverage or the like, in addition to those described above. In addition to the above, the fermented extract of the Perilla frutescens according to the present invention may contain fruit pulp for the preparation of natural fruit juices, fruit juice beverages and vegetable beverages. These ingredients may be used either alone or in combinations thereof. The proportion of these additives is not significantly important, but is generally selected within a range of 0.1 to 20 parts by weight per 100 parts by weight of the fermented extract of Perilla frutescens according to the present invention.

In addition, the present invention may provide a method for treating cognitive ability or memory ability-related disease, including administering a fermented extract of Perilla frutescens to a patient with cognitive ability or memory ability-related disease.

Since the fermented extract of Perilla frutescens is the same as that used in the composition for improving cognitive ability or memory ability, the description thereof is replaced with the above-described description.

Since the cognitive ability or memory ability-related disease is the same as the cognitive ability or memory ability related disease targeted in the pharmaceutical composition, the description thereof is replaced with the above-described description.

### Advantageous Effects

The fermented extract of Perilla frutescens according to the present invention can be effectively used as a composition for improving cognitive ability or memory ability since it significantly improves cognitive ability or memory ability compared to an unfermented extract of Perilla furescens. Furthermore, the fermented extract of Perilla frutescens according to the present invention can be effectively used in a composition or method for preventing, ameliorating or treating cognitive ability or memory ability-related disease.

### DESCRIPTION OF DRAWINGS

FIG. 1 shows the results of measuring the spontaneous alteration of mice through the Y-maze test after administration of an extract of Perilla frutescens (PFW) or fermented extracts of Perilla frutescens (PFB, PFL) to mice whose cognitive ability had been reduced (Restraint stress) due to restraint. It was confirmed that the spontaneous alteration was significantly increased in the case of administration of PFB or PFL compared to PFW, respectively.

FIG. 2 shows the results of measuring the spontaneous alteration of mice through the Y-maze test after administration of an extract of Perilla frutescens (PFW) or fermented extracts of Perilla frutescens (PFB, PFL) to mice whose cognitive ability had been reduced by the administration of scopolamine. It was confirmed that the spontaneous alteration was significantly increased in the case of administration of PFB or PFL compared to PFW.

### MODES OF THE INVENTION

### Example 1

Preparation of Perilla frutescens Extract and Fermented Product Thereof

Perilla frutescens was extracted with hot water at 80° C. and then sterilized at 121° C. for 20 minutes to prepare a hot water extract of Perilla frutescens (PFW). The sterilized hot water extract of Perilla frutescens was fermented with bacillus or lactobacillus to prepare a fermented extract of Perilla frutescens.

Specifically, Bacillus subtilis as the bacillus or Lactobacillus rhamnosus as the lactobacillus were inoculated into the Perilla frutescens extract, respectively, and it was fermented for 5 days while shaking culture at 140 rpm in an environment of 25° C. and 80% humidity to finally prepare fermented extracts of Perilla frutescens of a Bacillus subtilis-fermented product of Perilla frutescens (PFB) and a Lactobacillus rhamnosus-fermented product of Perilla frutescens (PFL).

### Example 2

### Induction of Deterioration of Cognitive Ability in Mice

### <2-1> Induction of Deterioration of Cognitive Ability by Restraint

100 mg/kg of 0.9% physiological saline (CTL, Veh), the Perilla frutescens extract (PFW) prepared in [Example 1] or the fermented extracts thereof (PFB, PFL) was orally administered to ICR mice (Orient Bio Co., Ltd.) once daily for 5 days, and 30 minutes after the administration, the mice were placed in a circular restraint frame for 3 hours each to induce the deterioration of cognitive ability. After the end of inducing the deterioration of cognitive ability, the mice were stabilized for 24 hours, and then, the Y-maze test was performed to measure the cognitive ability of each mouse.

### <2-2> Induction of Deterioration of Cognitive Ability by Scopolamine

100 mg/kg of 0.9% physiological saline (CTL, Veh), the Perilla frutescens extract (PFW) prepared in [Example 1] or the fermented extracts thereof (PFB, PFL) was orally administered to ICR mice (Orient Bio Co., Ltd.) once, and 30 minutes after the administration, scopolamine, which is an anticholinergic drug, was intraperitoneally administered at 1 mg/kg to induce the deterioration of cognitive ability. Upon 30 minutes after the administration of scopolamine, the Y-maze test was performed to measure the spatial cognitive ability of each mouse.

### Example 3

Confirmation of Effects of Fermented Extracts of Perilla frutescens

It was attempted to confirm whether the fermented extracts of Perilla frutescens are effective for mice in which the deterioration of cognitive ability was induced in Example 2 by the Y-maze test.

Specifically, the experimental apparatus for the Y-maze test consisted of a Y-shaped maze with four sides blocked and made of acrylic plates (3 cm wide, 36 cm long, 15 cm high), and each maze was placed at a constant angle of 120° from each other. After dividing the Y-shaped maze into areas A, B and C, the experimental animals were carefully placed in one area and allowed to move freely for 7 minutes, and then, the area where the experimental animals entered was observed and recorded. Afterwards, the number and sequence of entering each maze were measured to evaluate spontaneous alteration. When the mice entered three different areas sequentially (change behaviors, alteration), it was counted as 1 point, and when the mice did not consecutively enter, it was not counted as a score. The spontaneous alteration (%) was calculated by the following mathematical formula.

Spontaneous alteration (%)=(Total number of change behaviors)/(Total number of entries−2)×100 Mathematical Formula

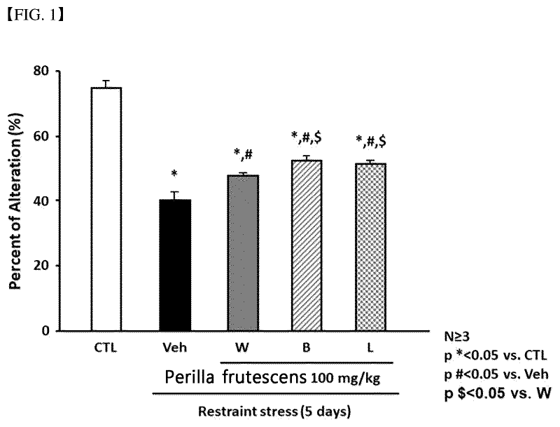

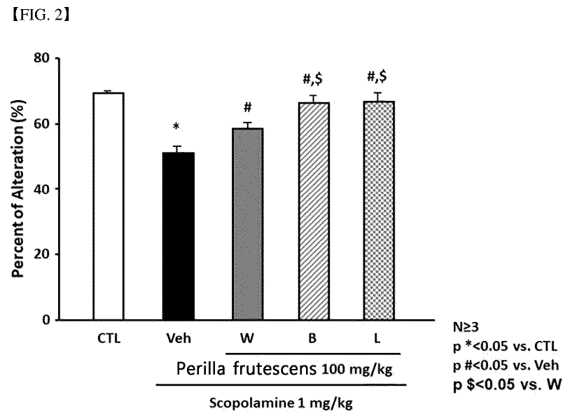

As a result, as shown in [FIG. 1] to [FIG. 2], the cognitive ability of the mice was significantly reduced by restraint or the administration of scopolamine. Accordingly, it was confirmed that when the Perilla frutescens extract (PFW) was administered, the cognitive ability of the mice was improved, and when the fermented extract of Perilla frutescens (PFB or PFL) was administered, the cognitive ability was significantly improved.

### Preparation Example

### 1. Preparation of Health Functional Formulation (Capsules)

| | | |
|---|---|---|
| Lactose | 25.0 | wt. % |
| Starch | 25.0 | wt. % |
| Talc | 8.0 | wt. % |
| Magnesium stearate | 2.0 | wt. % |
| Fermented extract of Perilla frutescens | 40.0 | wt. % |

The above ingredients were mixed and filled into gelatin capsules according to a conventional capsule preparation method to prepare capsules.

### 2. Preparation of Health Functional Beverage

| | | |
|---|---|---|
| Vitamin | 0.5 | wt. % |
| Dietary fiber | 4.0 | wt. % |
| Liquid fructose | 93.0 | wt. % |
| Emulsifier | 1.0 | wt. % |
| Fragrance | 0.5 | wt. % |
| Fermented extract of Perilla frutescens | 1.0 | wt. % |

Purified water was mixed with the composition such that a total volume was 50 mL. After passing the mixed solution through a 2 to 3 μm filter to remove the suspended matter, the final mixture was sterilized at 90 to 93° C. for 15 to 20 seconds and filled into a 50 mL bottle, and it was post-sterilized for 15 to 20 minutes at 80 to 85° C. to complete a health functional beverage product.

### 3. Preparation of Skin (Face Lotion)

| | | |
|---|---|---|
| Hydroxyethylene cellulose (2% aqueous solution) | 12.0 | wt. % |
| Xanthan gum (2% aqueous solution) | 2.0 | wt. % |
| 1,3-Butylene glycol | 6.0 | wt. % |
| Glycerin | 4.0 | wt. % |
| Sodium hyaluronate (1% aqueous solution) | 5.0 | wt. % |
| Ion-exchanged water | 68.0 | wt. % |
| Fermented extract of Perilla frutescens | 3.0 | wt. % |

The above ingredients were mixed to prepare a face lotion by a conventional face lotion preparation method.

### 4. Preparation of Fragrance

| | |
|---|---|
| 95% Ethanol | 65.0~75.0 wt. % |
| Fermented extract of Perilla frutescens | 25.0~35.0 wt. % |

The ethanol and the fermented extract of Perilla frutescens were mixed and stirred at room temperature for 12 to 20 minutes to prepare a fragrance.

### 5. Preparation of Bathing Agent

| | | |
|---|---|---|
| Sodium hydrogen carbonate | 70.0 | wt. % |
| Anhydrous sodium sulfate | 29.0 | wt. % |
| Fermented extract of Perilla frutescens | 1.0 | wt. % |

The sodium hydrogen carbonate and anhydrous sodium sulfate were stirred in a V-type mixer until becoming uniform, and then, the fermented extract of Perilla frutescens was added and sufficiently stirred until becoming uniform again to prepare a bathing agent.

### INDUSTRIAL APPLICABILITY

Since the fermented extract of Perilla frutescens according to the present invention significantly improves cognitive ability or memory ability compared to an unfermented extract of Perilla frutescens, it can be effectively used in a composition for improving cognitive ability or memory ability or a composition and method for preventing, ameliorating or treating cognitive ability or memory ability-related disease, and thus, it has high industrial applicability.

1. A composition for improving cognitive ability or memory ability, comprising a fermented extract of Perilla frutescens.

2. The composition of claim 1, wherein the fermented extract is extracted from any one or more parts selected from the group consisting of leaves, stems, flowers, fruits and whole plants of Perilla frutescens.

3. The composition of claim 1, wherein the fermented extract is obtained by extracting with water, an organic solvent or a mixture thereof as a solvent and then fermenting with a fermentation strain.

4. The composition of claim 3, wherein the fermentation strain is composed of bacillus or lactobacillus.

5. The composition of claim 4, wherein the bacillus is any one or more Bacillus genus bacteria selected from the group consisting of Bacillus subtilis, Bacillus thuringiensis, Bacillus licheniformis, Bacillus amyloliquefaciens, Bacillus stearothermophilus, Bacillus coagulans, Bacillus longum, Bacillus pumilus, Bacillus brevis, Bacillus circulans and Bacillus polymyxa; and
wherein the lactobacillus is any one or more Lactobacillus genus bacteria selected from the group consisting of Lactobacillus acidophilus, Lactobacillus casei, Lactobacillus gasseri, Lactobacillus bulgaricus, Lactobacillus helveticus, Lactobacillus fermentum, Lactobacillus paracasei, Lactobacillus plantarum, Lactobacillus reuteri, Lactobacillus rhamnosus and Lactobacillus salivarius.

6. The composition of claim 3, wherein the fermentation is performed at 5° C. to 80° C. for 30 minutes to 10 days.

7. The composition of claim 1, wherein the composition is cosmetic composition.

8. A fiber for improving cognitive ability or memory ability, comprising a fermented extract of Perilla frutescens.

9. The fiber of claim 8, wherein the fiber is dyed with a fermented extract of Perilla frutescens.

10. The fiber of claim 8, wherein the fiber is included in any one or more products selected from the group consisting of bedding, clothing, curtains, carpets, indoor shoes, towels, wallpaper, indoor cover products made of textile materials, dolls and eye masks.

11. A fragrance composition for improving cognitive ability or memory ability, comprising a fermented extract of Perilla frutescens.

12. The fragrance composition of claim 11, wherein the fragrance composition is included in any one or more products selected from the group consisting of perfumes, scented candles, oils, aromatics, detergents and external skin preparations.

13. A pharmaceutical composition for preventing or treating cognitive ability or memory ability-related disease, comprising a fermented extract of Perilla frutescens.

14. The pharmaceutical composition of claim 13, wherein the cognitive ability or memory ability-related disease is degenerative brain disease or dementia.

15. A health functional food composition for preventing or ameliorating cognitive ability or memory ability-related disease, comprising a fermented extract of Perilla frutescens.

16. A method for treating cognitive ability or memory ability-related disease, comprising administering a fermented extract of Perilla frutescent to a patient with cognitive ability or memory ability-related disease.

17. The method of claim 16, wherein the fermented extract is extracted from any one or more parts selected from the group consisting of leaves, stems, flowers, fruits and whole plants of Perilla frutescens.

18. The method of claim 16, wherein the fermented extract is obtained by extracting with water, an organic solvent or a mixture thereof as a solvent and then fermenting with a fermentation strain.

19. The method of claim 18, wherein the fermentation strain is composed of bacillus or lactobacillus.

20. The method of claim 19, wherein the bacillus is any one or more Bacillus genus bacteria selected from the group consisting of Bacillus subtilis, Bacillus thuringiensis, Bacillus licheniformis, Bacillus amyloliquefaciens, Bacillus stearothermophilus, Bacillus coagulans, Bacillus longum, Bacillus pumilus, Bacillus brevis, Bacillus circulans and Bacillus polymyxa; and
wherein the lactobacillus is any one or more Lactobacillus genus bacteria selected from the group consisting of Lactobacillus acidophilus, Lactobacillus casei, Lactobacillus gasseri, Lactobacillus bulgaricus, Lactobacillus helveticus, Lactobacillus fermentum, Lactobacillus paracasei, Lactobacillus plantarum, Lactobacillus reuteri, Lactobacillus rhamnosus and Lactobacillus salivarius.
